(19) United States
(12) Patent Application Publication
(10) Pub. No.: US 2015/0361191 A1
(43) Pub. Date: Dec. 17, 2015

(54) A PROCESS FOR THE PREPARATION OF POLYETHYLENE WAX USING METALLOCENE CATALYST

(71) Applicants: Seung-Woo Ko, Gyeonggi-do (KR); Dong Wook Jeong, Daejeon (KR)

(72) Inventors: Seung-Woo Ko, Gyeonggi-do (KR); Dong Wook Jeong, Daejeon (KR)

(73) Assignee: HANWHA CHEMICAL CORPORATION, Seoul (KR)

(21) Appl. No.: 14/240,148

(22) PCT Filed: Aug. 13, 2012
(86) PCT No.: PCT/KR2012/006445

§ 371 Date: May 15, 2014

(30) Foreign Application Priority Data
Aug. 25, 2011 (KR) 10-2011-0084979

(51) Int. Cl. C08F 110/02; C07C 2/32

(52) U.S. Cl. CPC C08F 110/02; C07C 2/32; C07C 2531/22; C07C 2531/14

(57) ABSTRACT

The present invention relates to a method for preparing a polyethylene wax, comprising the step of polymerizing ethylene monomers using a metallocene catalyst in a loop reactor, and more particularly, to a method for polymerizing a polyethylene wax using a metallocene catalyst and a double loop reactor. According to the present invention, a polyethylene wax having a uniform and narrow molecular weight distribution can be polymerized with high activity.

### TECHNICAL FIELD

The present invention relates to a method for polymerizing a polyethylene wax using a metallocene catalyst system in a loop reactor, and more particularly, to a method for efficiently preparing a polyethylene wax according to particular polymerization conditions.

### BACKGROUND ART

Wax is a plastic solid at a low temperature, but becomes a low viscosity liquid when the temperature increases to approximately 100° C., and defined as an organic mixture or compound having alkyl groups (CₙH₂ₙ₊₁—) and a molecular weight of 500-10,000 g/mol. Wax has flammability and excellent insulativity of water- and moisture-proof, and is soluble in most organic solvents, but insoluble in water.

Wax is used in a wide variety of applications such as candles, paper and textile processing, electrical industries, civil engineering and construction, stationery, artistic handicrafts, rubber compounds, solid lubricants, adhesives, cosmetics, and medicines.

Polyethylene (PE) wax means a polyethylene having a weight-average molecular weight of 500-10,000 g/mol, and is a representative synthetic wax produced from ethylene. Polyethylene wax is classified into several different categories based on its preparation method, density, size, and state. In this regard, polyethylene polymers include wax, ultra high molecular weight polyethylene (UHMWPE) or the like, and the types are divided depending on their molecular weight. That is, the above substances largely belong to polyethylene polymers, but they have different characteristics depending on the molecular weight and thus their uses may differ from each other.

Among the polyethylene polymers, polyethylene wax has excellent compatibility and dispersibility with other base materials and also excellent electrical insulation properties and chemical resistance. Polyethylene wax is used for the purpose of viscosity control, quenching effect, surface texturing, water-proof, and rust prevention in a wide range of applications such as master batch, processing materials, hot melt adhesives, paints, coatings, inks or the like. In some applications, petroleum wax, natural wax, and other synthetic wax are substituted for polyethylene wax.

Meanwhile, polyethylene wax is divided into polymer wax, thermal cracking wax, and by-product wax according to preparation methods.

The polymer wax is subsequently divided into a high-pressure polyethylene wax produced by a high pressure process and a low-pressure polyethylene wax produced by a low pressure process using metallocene and Ziegler-Natta catalysts. It is also divided into a high-density PE wax having a density of 0.93 g/cc or higher and a low-density PE wax having a density of less than 0.93 g/cc according to its density.

Problematically, pyrolysis used for the preparation of thermal cracking wax is a complex process because it should be performed after polymerization of polyethylene, and it is also difficult to control the reaction and to obtain products having uniform quality because of a wide molecular weight distribution. In order to improve these problems, various studies have been made, but there are still difficulties in the control of reaction conditions.

In order to improve the problems in the PE wax preparation by pyrolysis, ethylene has been polymerized to have a low polymerization degree. In this method, hydrogen is widely used as a chain-transfer agent for the control of polymerization degree.

The molecular weight of polyethylene depends on the amount of hydrogen injected into a reactor. Hydrogen functions as a very effective chain-transfer agent in ethylene polymerization. However, because polymerization is performed in the presence of a large amount of hydrogen in order to reduce the molecular weight, addition of hydrogen to ethylene allows side reaction of producing ethane, and thus activity is reduced, resulting in a low yield of PE wax polymerization. Moreover, the use of Ziegler-Natta catalyst and hydrogen in the preparation of polyethylene wax causes problems of producing a considerable amount of oligomers and broadening the molecular weight distribution.

Therefore, use of metallocene catalysts has been studied to solve these problems. It is possible to prepare a polyethylene wax having a narrow molecular weight distribution by using metallocene catalysts, owing to the single site characteristic of metallocene catalysts with every catalyst site active for polymerization being identical. Therefore, metallocene polyethylene wax shows a narrow molecular weight distribution and high crystallinity, unlike the common polyethylene wax.

The use of metallocene catalysts and preparation methods of wax are exemplified by U.S. Pat. No. 4,914,253, Korean Patent No. 0137960, U.S. Pat. No. 5,750,813, Korean Patent Nos. 0310933 and 0615460.

However, these methods are still problematic in terms of catalyst efficiency and durability.

### DISCLOSURE

### Technical Problem

In order to solve the above problems, an object of the present invention is to provide a method for preparing a polyethylene wax with excellent activity and controllable molecular weight distribution using a metallocene catalyst system in a loop reactor.

However, the object to be achieved in the present invention is not limited to those described above and other objects will be clearly understood by those skilled in the art from the following description.

### Technical Solution

In order to achieve the above object, the present invention provides a method for preparing a polyethylene wax, comprising the step of polymerizing ethylene monomers in the presence of a metallocene catalyst in a loop reactor.

In one embodiment of the present invention, the loop reactor is a double loop reactor composed of a first reactor and a second reactor connected to each other.

In one embodiment of the present invention, a solvent of isobutane, normal hexane, or a mixture thereof may be further used in the method.

In one embodiment of the present invention, the metallocene catalyst includes a metallocene catalyst that is represented by the following Chemical Formula 1.

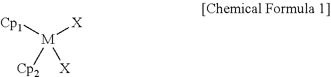

wherein M is a metal atom selected from titanium (Ti), zirconium (Zr), and hafnium (Hf), and Cp₁ and Cp₂ are each independently a cyclopentadienyl, indenyl or fluorenyl group; and X is a halogen atom, a C₁˜C₁₀ alkyl group, or a C₆˜C₂₀ aryl group.

In one embodiment of the present invention, the metallocene catalyst may further include an aluminium cocatalyst.

In one embodiment of the present invention, the metallocene catalyst preferably has a molar ratio of aluminium of the aluminium cocatalyst to the metal of the Chemical Formula 1 of 1:500-1:2000.

In one embodiment of the present invention, the aluminium cocatalyst may be alkyl aluminoxane where a C₁-C₅ alkyl group is connected to aluminium.

In one embodiment of the present invention, the metallocene catalyst may be an unsupported or supported catalyst.

In one embodiment of the present invention, a support used in the supported catalyst may be selected from the group consisting of silica, alumina, magnesium chloride, zeolite, aluminium phosphate, and zirconia.

In one embodiment of the present invention, the method may be performed under the conditions of a polymerization temperature of 50-90° C., a hydrogen injection of 10% or less, a maximum reactor available pressure of 20-35 kg/cm², a maximum ethylene available pressure of 10 kg/cm², a polymerization time of 30 minutes or longer, and preferably for 30˜180 minutes.

In one embodiment of the present invention, the support used in the supported catalyst is silica, and the silica is preferably dehydrated silica having a specific surface area of 50 m²/g-500 m²/g, and a hydroxyl group of 0.5-3 number/cm².

In one embodiment of the present invention, a method for preparing a polyethylene wax in the double loop reactor composed of a first reactor and a second reactor connected to each other comprises the steps of polymerizing ethylene monomers and hydrogen in the presence of a metallocene catalyst and a solvent in a first reactor; polymerizing a product produced by the above step and a solvent in a second reactor; and separating a product of the second reactor in a separator.

In one embodiment of the present invention, the polymerization method further comprises the step of reusing the solvent separated by the separator in the first and second reactors.

In one embodiment of the present invention, the polymerization method further comprises the step of activating the catalyst by reaction of the metallocene catalyst and the cocatalyst, prior to the reaction step in the first reactor.

Other embodiments of the present invention are included in the following detailed description.

### Advantageous Effects

According to the present invention, PE wax having a narrow molecular weight distribution and excellent quality can be polymerized with high activity by using a metallocene catalyst and a loop reactor.

### DESCRIPTION OF DRAWINGS

FIG. 1 is a schematic view showing a process of double loop reactor connected in serial according to the present invention;

FIG. 2 is a schematic view showing a production process of polyethylene wax according to one embodiment of the present invention; and

FIG. 3 is a graph showing solvent evaporation points according to monomer content.

### BEST MODE

The present inventors have made many efforts to prepare a polyethylene wax in a loop reactor. As a result, they found that a polyethylene wax having a uniform and narrow molecular weight distribution can be prepared with excellent activity by polymerization of ethylene and hydrogen using a metallocene catalyst.

The metallocene catalyst means a metallocene catalyst compound that may include a metallocene catalyst of the following Chemical Formula 1, and may further include a cocatalyst, a support or a mixture thereof.

According to one embodiment of the present invention, provided is a method for preparing a polyethylene wax, including the step of polymerizing ethylene monomers in the presence of a metallocene catalyst in a loop reactor.

According to one embodiment of the present invention, the loop reactor may be preferably a double loop reactor composed of a first reactor and a second reactor connected to each other.

That is, the process of the present invention is characterized in that the polyethylene wax is prepared by using the metallocene catalyst and the double loop reactor at the same time. Hydrogen reactivity differs depending on the characteristics of the metallocene catalyst, and thus a wax having excellent physical properties can be prepared according to the characteristics of catalyst.

Preparation methods of polyethylene wax are exemplified by gas phase polymerization, liquid polymerization, and slurry polymerization. In these methods, a gas-phase reactor, a loop reactor, a double loop reactor, and a CSTR reactor are used.

In the double loop reactor, medium to high density-polyethylene products are mainly produced, and LLDPE can be also produced. The greatest advantage of this method is to produce polyethylene having a medium molecular weight distribution (Mw/Mn=10˜20), which is the most suitable for blow molding.

An upper limit of the slurry concentration inside the reactor should not affect the fluid behavior inside the reactor and should ensure effective heat transfer efficiency through the reactor wall. In the process, temperature is one of the most important operation variables, and should be controlled in the range of 0.1° C. According to one embodiment of the present invention, the conversion rate of the monomers is 98˜99%.

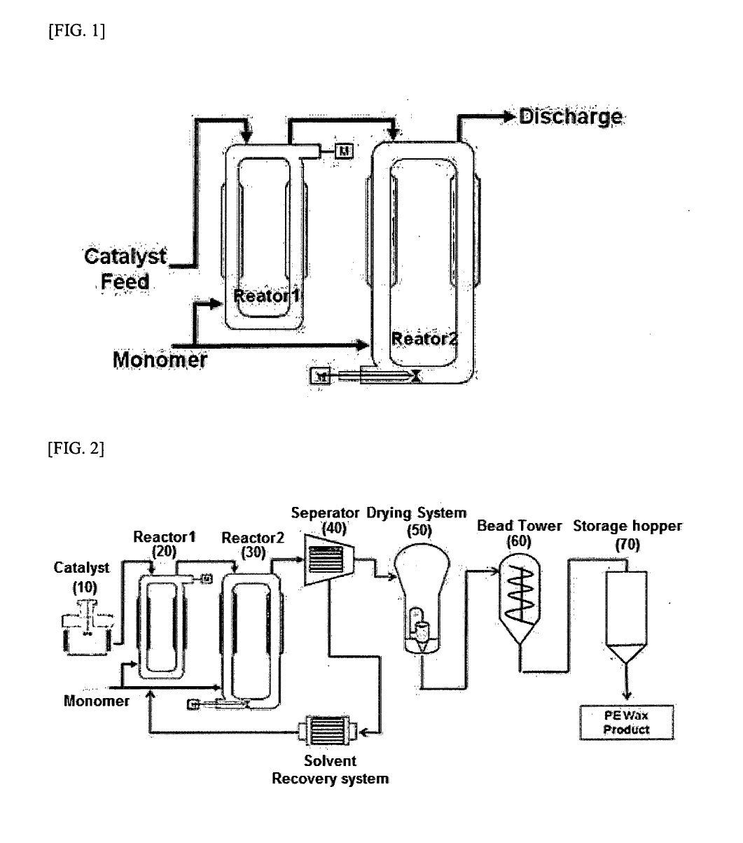

The double loop reactor according to the present invention is illustrated in FIG. 1.

With reference to FIG. 1, the catalyst activated by mixing the catalyst and the cocatalyst is injected into the first reactor, and reacted with monomers to cause polymerization. Polymer particles that grow with the solvent circulation during the reaction are transported to the second reactor to complete the polymerization.

In one aspect of the present invention, the polymerization method of the polyethylene wax is performed in the double loop reactor composed of the first reactor and the second reactor connected to each other, the method including the steps of polymerizing ethylene monomers and hydrogen in the presence of the metallocene catalyst and the solvent in the first reactor; polymerizing a product produced by the above step and the solvent in the second reactor; and separating a product of the second reactor in a separator.

The polymerization method further includes the step of reusing the solvent separated by the separator in the first and second reactors.

The method may be performed under the conditions of a polymerization temperature of 50-90° C., a hydrogen injection of 10% or less, a maximum reactor available pressure of 20-35 kg/cm², a maximum ethylene available pressure of 10 kg/cm² or less, and a polymerization time of 30˜180 minutes.

In the method, one or more comonomers selected from the group consisting of 1-butene, 1-pentene, 1-hexene, 4-methyl-1-pentene, 1-octene, 1-decene, 1-dodecene, 1-tetradecene, 1-hexadecene, 1-octadecene, and mixtures thereof may be further used during polymerization.

In one embodiment of the present invention, the polymerization method may further include the step of activating the catalyst by reaction of the metallocene catalyst and the cocatalyst, prior to the reaction step in the first reactor.

The preparation process of the polyethylene wax using the loop reactor of the present invention is illustrated in FIG. 2.

With reference to FIG. 2, the residence time is controlled by mileage control of approximately 100 kg Polymer/1 kg Catalyst in the first reactor, followed by transport into the second reactor. In the second reactor, the residence time is controlled to approximately 900 kg Polymer/1 kg Catalyst to complete the polymerization reaction. While passing through the first and second reactors, the catalysts and monomers slowly react with each other and polymers grow. Physical properties of the product are controlled according to the injection amount of hydrogen, polymerization temperature, and reaction time.

Further, the liquid phase solvent in the loop reactor may be changed into a liquid or gas state according to polymerization temperature when it enters the reactor. However, it is important to be in a full liquid state, because the loop reactor should be filled with the liquid phase solvent for circulation. The evaporation point of the solvent in the reactor changes according to the amount of monomers, and pressure and temperature of the reactor, and thus it is important that the amount of monomers, and pressure and temperature of the reactor are controlled to be operable in a full liquid state.

As shown in FIG. 2, the catalyst and the cocatalyst 10 are mixed at a predetermined ratio for activation, and then injected into the first reactor 20 (Reactor 1). When the pressure and temperature are set up to maintain the solvent in the full liquid state, and monomers (ethylene) and hydrogen are injected while the solvent is circulated by a motor, if necessary, comonomers (butene, hexene) is added, reaction is initiated. The polymer particles formed while circulating in the reactor for 30 minutes are transported to the second reactor 30 (Reactor 2), and the reaction is continued. In the second reactor 30, the polymer particles are formed while the solvent is circulated for approximately 60 minutes, and then transported into a separator 40 to separate the particles and the solvent. The separated solvent is re-injected into the first reactor 20 and the second reactor 30, and the particles are transported into a drying system 50. In the drying system, the particles are dried at a high temperature, and the solvent, residual catalyst, and monomers are completely removed, and then transported into a bead tower 60. In the bead tower 60, the polymers in non-uniform polymer particles are melted to produce bead-type products, which are transported into a storage hopper 70, followed by storage and manufacture. In the case of a support system, polymers are formed in spherical particles, and thus may be used as it is without passing through the bead tower 60.

The solvent used in the polymerization of the present invention is not particularly limited, but isobutane, normal hexane, or a mixture thereof may be preferably used.

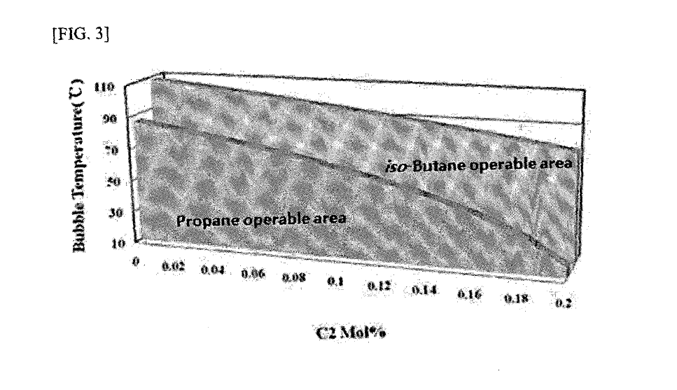

In this regard, the solvent commonly used in the loop reactor may be isobutane, propane, pentane or the like. However, the results of calculating the evaporation point of the solvent according to the monomer content, as shown in FIG. 3, showed that the polymerization temperature and the hydrogen content limit the operable temperature. Thus, a more preferred solvent is isobutane or normal hexane that has a wide operable temperature range upon polymerization of polyethylene wax.

Further, the metallocene catalyst preferably includes a metallocene catalyst represented by the following Chemical Formula 1.

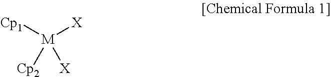

wherein M is a metal atom selected from titanium (Ti), zirconium (Zr), and hafnium (Hf), and Cp₁ and Cp₂ are each independently a cyclopentadienyl, indenyl or fluorenyl group; and X is a halogen atom, a C₁˜C₁₀ alkyl group, or a C₆˜C₂₀ aryl group.

The metallocene catalyst used in the polymerization of the present invention may further include a cocatalyst, and preferably, an aluminium cocatalyst.

In the metallocene catalyst used in the polymerization reaction, a molar ratio of aluminium of the aluminium cocatalyst to the metal of the Chemical Formula 1 is preferably 1:500-1:2000. If it is not within the above range, activity is too low to induce the polymerization, or overreaction occurs, which makes it difficult to find operation conditions.

The aluminium cocatalyst is preferably aluminium connected with an alkyl group, and more preferably, alkyl aluminoxane where a C₁-C₅ alkyl group is connected to aluminium.

The metallocene catalyst is an unsupported or supported catalyst.

A support used in the supported catalyst may be selected from the group consisting of silica, alumina, magnesium chloride, zeolite, aluminium phosphate, and zirconia.

The support used in the metallocene catalyst of the present invention is preferably silica.

If the support used in the supported catalyst is silica, the silica is preferably dehydrated silica having a specific surface area of 50 m²/g-500 m²/g, and a hydroxyl group of 0.5-3 number/cm², but is not limited thereto.

The polymerization reaction may be performed under the conditions of a polymerization temperature of 50-90° C., a hydrogen injection amount of 10% or less, and a polymerization time of 30 minutes or longer. More preferably, the hydrogen injection amount may be 0% or more to 10% or less, and the polymerization time may be 30 minutes to 180 minutes. Herein, if the hydrogen injection amount exceeds 10%, there are problems that the reaction is terminated by hydrogen as a chain-transfer agent, and thus the activity becomes low and the polyethylene wax has a very low molecular weight. In addition, if the polymerization time is less than 30 minutes, the reaction is early terminated, and thus it is difficult to obtain a polyethylene wax having a desired molecular weight in a high yield.

Further, a maximum available pressure of the loop reactor of the present invention is preferably 20-35 kg/cm², and a maximum ethylene available pressure is preferably 10 kg/cm². When the supported metallocene catalyst is used, the maximum ethylene available pressure is more preferably 10 kg/cm² or less, and when the unsupported metallocene catalyst is used, the maximum ethylene available pressure is more preferably 7 kg/cm² or less

If the conditions are satisfied, the solvent completely becomes in a liquid state in the loop reactor. In addition, if the reactor available pressure exceeds the above range, it creates safety problems in the reactor, and if the ethylene available pressure exceeds the above range, the injection amount of hydrogen should be increased depending on the increased ethylene available pressure, which makes it difficult to determine the operating conditions.

When the polymerization temperature is not within the above range, the catalytic activity is reduced. When the injection amount of hydrogen is not within the range of 10% or less, there are problems that the activity is reduced and the produced polyethylene wax has a very low molecular weight.

### MODE FOR INVENTION

Hereinafter, the embodiments of the present invention will be described in detail. However, these are for illustrative purposes only, and the present invention is not intended to be limited thereto. The present invention will only be defined by the appended claims.

### Preparation Example 1

### Catalyst Synthesis: Bis(indenyl)ZrCl₂

After dilution of indene in THF, the temperature was reduced to −78° C. Bath, and n-BuLi was slowly injected. Thereafter, the temperature was increased to room temperature by removal of the low temperature bath, followed by stirring for 5 hours. A Li salt formed as a powder was filtered to obtain indenyl lithium. Indenyl lithium and ZrCl₄ were weighed, and then put in a flask, and THF was injected thereto, followed by stirring. After 5 hours, the Li salt was removed, and the resultant was mixed with the prepared indenyl lithium, followed by stirring under THF. After 5 hours, LiCl was removed by filtration, and the solvent was also removed to obtain a yellow oil product.

### Preparation Example 2

### Catalyst Synthesis: (n-BuCp)₂ZrCl₂

Dicyclopentadiene was cracked at 180° C. Cyclopentadiene was diluted in THF, and the temperature was reduced to −78° C. Then, bromobutane was slowly injected, followed by stirring for 12 hours. After completing the injection of bromobutane, the low bath was removed, and the reaction was allowed at room temperature. Thereafter, THF was removed from the reactant, and bromocyclopentadiene was prepared by extraction with pentane. The bromocyclopentadiene was diluted in THF, and the temperature was reduced to −78° C. Then, n-BuLi(2.5 M/n-hexane) was injected, and the temperature was raised to room temperature, followed by stirring for 5 hours. After removal of the solvent, the resultant was washed with pentane to obtain white powder. Two equivalents of the powder and one equivalent of ZrCl₄ were put in a flask, and cold (−30° C.) toluene was immediately injected, followed by stirring for 2 hours. Subsequently, toluene was removed, and a catalyst was obtained by trituration with pentane.

### Preparation Example 3

### Preparation of Supported Catalyst

Support of MAO(Methylaluminoxane) on Silica

2 g of Grace silica XPO-2402 (average particle size: 50 μm) was suspended in toluene to prepare a silica slurry.

1 mmol of the catalyst of Preparation Example 1 was put in a separate reactor, and 9.275 ml of methylaluminoxane (10 wt % MAO in toluene, Albemarle Corporation) as a cocatalyst was added at 30° C., and then stirred for approximately 30 minutes to obtain an activated catalyst. Thereafter, the activated catalyst solution was slowly injected to the silica slurry at room temperature, and stirred for 2 hours. After termination of the stirring, the supernatant was removed, and the resultant was washed with 10 ml of hexane, and dried under vacuum to prepare a silica-supported catalyst.

### Preparation Example 4

### Preparation of Supported Catalyst

A supported catalyst was prepared in the same manner as in Preparation Example 3, except that the catalyst of Preparation Example 2 was used instead of the catalyst of Preparation Example 1.

### EXAMPLES

In the following polymerization method of ethylene, a 2 L autoclave reactor was used, and a catalyst, a cocatalyst, ethylene, and hydrogen were injected to the reactor. Then, a polymerization reaction was allowed while maintaining a predetermined pressure, and isobutane or normal hexane was used as a medium. At this time, because a double loop reactor was a commercial reactor, polymerization evaluation was not carried out, and operating conditions were calculated through simulation. As described in Detailed Description, the process constitutions of FIGS. 1 and 2 were designed. That is, even though performed in the autoclave reactor, the reaction can mimic solution and slurry polymerizations, and the scale of commercial reactor can be predicted therefrom.

Further, the following properties of each polymer were determined by the following methods.

Number-average molecular weight (Mn), weight-average molecular weight (Mw), and molecular weight distribution (MWD) were determined by Gel Permeation Chromatograpy (GPC) after dissolving each polymer in 1,2,4-trichlorobenzene.

Melt viscosity was determined using a Brookfield viscometer in accordance with ASTM D2669-87.

Softening Point was determined using a softening point tester in accordance with ASTM D2669-06.

Meting point (Tm) was determined using a differential scanning calorimeter (DSC).

Density was determined using an auto density meter, and measurement was repeated four times for each sample to determine mean values.

In the following Examples 1-6, 13-18, 25-30, and 37-42, isobutane was used as a solvent, and in the following Examples 7-12, 19-24, 31-36, and 43-48, normal hexane was used as a solvent. A polymerization solvent, isobutane or hexane was passed through a molecular sieve dried at high temperature for removal of impurities, followed by storage until use.

### 1. Examples Using Unsupported Catalyst

### Example 1

An autoclave reactor, made from a metal, with an internal volume of 2 L was used, and nitrogen was substituted for the gas inside the reactor before initiation of polymerization. The reactor was heated to high temperature, and then maintained under vacuum.

The reactor was filled with 1.2 L of a solvent (isobutane), followed by injection of the activated catalyst (metallocene catalyst) prepared by mixing methylaluminoxane [MAO(Albermale, 10 wt % toluene)] and the catalyst (Bis(indenyl)ZrCl₂) of Preparation Example 1 based on a molar ratio of Al/M=1:1000. Then, polymerization reaction was performed for 30 minutes while maintaining the polymerization temperature of 55° C., the ethylene injection of 4 kg/cm², the initial hydrogen injection of 500 ml, and the H₂/C₂ ratio of 2%.

After termination of the polymerization, the reactor temperature was reduced to room temperature, and the solvent was separated using a separator so as to recover the polymer and solvent. Then, the polymer was dried in a vacuum oven at 50° C. for 6 hours. Polymerization of polyethylene wax was completed through this procedure.

Polymerization results according to the solvent, H₂/C₂ ratio, and polymerization temperature are shown in Table 1.

Polymerization activity (g-PE/g-cat,hr) was calculated from a weight ratio of the polymer produced per the catalyst used (g).

### Example 2

Ethylene polymerization was performed in the same manner as in Example 1, except that the polymerization was performed while maintaining the H₂/C₂ ratio of 3%.

### Example 3

Ethylene polymerization was performed in the same manner as in Example 1, except that the polymerization was performed while maintaining the H₂/C₂ ratio of 4%.

### Example 4

Ethylene polymerization was performed in the same manner as in Example 1, except that the polymerization was performed while maintaining the polymerization temperature of 60° C.

### Example 5

Ethylene polymerization was performed in the same manner as in Example 4, except that the polymerization was performed while maintaining the H₂/C₂ ratio of 3%.

### Example 6

Ethylene polymerization was performed in the same manner as in Example 4, except that the polymerization was performed while maintaining the H₂/C₂ ratio of 4%.

### Example 7

Polyethylene wax was prepared in the same manner as in Example 1, except that normal hexane was used as the solvent.

### Example 8

Ethylene polymerization was performed in the same manner as in Example 7, except that the polymerization was performed while maintaining the H₂/C₂ ratio of 3%.

### Example 9

Ethylene polymerization was performed in the same manner as in Example 7, except that the polymerization was performed while maintaining the H₂/C₂ ratio of 4%.

### Example 10

Ethylene polymerization was performed in the same manner as in Example 7, except that the polymerization was performed while maintaining the polymerization temperature of 60° C.

### Example 11

Ethylene polymerization was performed in the same manner as in Example 10, except that the polymerization was performed while maintaining the H₂/C₂ ratio of 3%.

### Example 12

Ethylene polymerization was performed in the same manner as in Example 10, except that the polymerization was performed while maintaining the H₂/C₂ ratio of 4%.

TABLE 1
Polymerization results with unsupported catalyst of Preparation
Example 1

| | | H₂/C₂ | Activity | | | | | Softening | |
|---|---|---|---|---|---|---|---|---|---|
| | Temperature | Ratio | g-PE/ | Mw | | Tm | Viscosity | Point | Density |
| Example | ° C. | % | g-Cat · hr | g/mol | MWD | ° C. | mPa · s | ° C. | g/cc |
| | Solvent: iso-butane | | | | | | | | |
| 1 | 55 | 2 | 145000 | 4670 | 4.26 | 123 | 910 | 133 | 0.95 |
| 2 | 55 | 3 | 137000 | 3492 | 3.19 | 122 | 188 | 129 | 0.95 |
| 3 | 55 | 4 | 132000 | 2570 | 3.16 | 121 | 86 | 127 | 0.95 |
| 4 | 60 | 2 | 145000 | 4824 | 4.49 | 124 | 132 | 132 | 0.95 |
| 5 | 60 | 3 | 140000 | 4127 | 4.03 | 122 | 127 | 127 | 0.95 |
| 6 | 60 | 4 | 138000 | 2446 | 2.65 | 121 | 126 | 126 | 0.95 |
| | Solvent: n-hexane | | | | | | | | |
| 7 | 55 | 2 | 278000 | 8928 | 3.81 | 126 | 1400 | 135 | 0.96 |
| 8 | 55 | 3 | 256000 | 3203 | 2.66 | 120 | 560 | 131 | 0.95 |
| 9 | 55 | 4 | 226000 | 2336 | 2.28 | 118 | 38 | 126 | 0.94 |
| 10 | 60 | 2 | 380000 | 8283 | 4.62 | 126 | 970 | 130 | 0.96 |
| 11 | 60 | 3 | 298000 | 3894 | 3.78 | 121 | 53 | 127 | 0.95 |
| 12 | 60 | 4 | 270000 | 2247 | 2.78 | 119 | 10 | 121 | 0.94 |

*Polymerization conditions: partial pressure of ethylene = 4 kg/cm², initial hydrogen injection = 500 ml, polymerization time = 30 minutes, [Al]/[M]Ratio = 1/1000

As shown in the results of Table 1, different physical properties were exhibited according to the medium. However, as the polymerization temperature was increased, the activity was increased, and as the hydrogen content was increased, the molecular weight, molecular weight distribution, and viscosity were reduced.

In addition, when hexane was used as the solvent, high activity, molecular weight, molecular weight distribution, and viscosity were observed, compared to the use of isobutane. When isobutane was used, the activity was reduced, but physical properties of wax could be easily controlled, and drying and separation processes could be also easily performed, compared to the use of hexane.

### Example 13

Ethylene polymerization was performed in the same manner as in Example 1, except that the polymerization was performed using 10 μmol of Bis(n-butylcyclopentadienyl)ZrCl₂ catalyst of Preparation Example 2.

### Example 14

Ethylene polymerization was performed in the same manner as in Example 13, except that the polymerization was performed while maintaining the H₂/C₂ ratio of 3%.

### Example 15

Ethylene polymerization was performed in the same manner as in Example 13, except that the polymerization was performed while maintaining the H₂/C₂ ratio of 4%.

### Example 16

Ethylene polymerization was performed in the same manner as in Example 13, except that the polymerization was performed while maintaining the polymerization temperature of 60° C.

### Example 17

Ethylene polymerization was performed in the same manner as in Example 16, except that the polymerization was performed while maintaining the H₂/C₂ ratio of 3%.

### Example 18

Ethylene polymerization was performed in the same manner as in Example 16, except that the polymerization was performed while maintaining the H₂/C₂ ratio of 4%.

### Example 19

The reactor was filled with 1.2 L of a solvent (normal hexane), followed by injection of the activated catalyst prepared by mixing methylaluminoxane [MAO(Albermale, 10 wt % toluene)] and the catalyst (n-BuCp)₂ZrCl₂) of Preparation Example 2 based on a molar ratio of Al/M=1:1000.

### Example 20

Ethylene polymerization was performed in the same manner as in Example 19, except that the polymerization was performed while maintaining the H₂/C₂ ratio of 3%.

### Example 21

Ethylene polymerization was performed in the same manner as in Example 19, except that the polymerization was performed while maintaining the H₂/C₂ ratio of 4%.

### Example 22

Ethylene polymerization was performed in the same manner as in Example 19, except that the polymerization was performed while maintaining the polymerization temperature of 60° C.

### Example 23

Ethylene polymerization was performed in the same manner as in Example 22, except that the polymerization was performed while maintaining the H₂/C₂ ratio of 3%.

### Example 24

Ethylene polymerization was performed in the same manner as in Example 22, except that the polymerization was performed while maintaining the H₂/C₂ ratio of 4%.

TABLE 2
Polymerization results with unsupported catalyst of Preparation
Example 2

| | | H₂/C₂ | Activity | | | | | Softening | |
|---|---|---|---|---|---|---|---|---|---|
| | Temperature | Ratio | g-PE/ | Mw | | Tm | Viscosity | Point | Density |
| Example | ° C. | % | g-Cat · hr | g/mol | MWD | ° C. | mPa · s | ° C. | g/cc |
| | Solvent: iso-butane | | | | | | | | |
| 13 | 55 | 2 | 112500 | 1128 | 3.44 | 124 | 7500 | 125 | 0.95 |
| 14 | 55 | 3 | 115800 | 1002 | 3.16 | 123 | 2800 | 126 | 0.95 |
| 15 | 55 | 4 | 110500 | 815 | 2.89 | 123 | 1800 | 125 | 0.95 |
| 16 | 60 | 2 | 113200 | 1942 | 4.56 | 128 | 9600 | 133 | 0.97 |
| 17 | 60 | 3 | 112000 | 1621 | 3.72 | 126 | 1430 | 129 | 0.96 |
| 18 | 60 | 4 | 111500 | 1176 | 2.82 | 124 | 680 | 129 | 0.96 |
| | Solvent: n-hexane | | | | | | | | |
| 19 | 55 | 2 | 146000 | 8670 | 5.41 | 131 | 9200 | 135 | 0.97 |
| 20 | 55 | 3 | 131000 | 7798 | 5.00 | 129 | 8300 | 132 | 0.97 |
| 21 | 55 | 4 | 114000 | 4608 | 4.99 | 126 | 2017 | 129 | 0.96 |
| 22 | 60 | 2 | 186000 | 5616 | 4.57 | 129 | 1400 | 131 | 0.96 |
| 23 | 60 | 3 | 148000 | 3476 | 3.81 | 128 | 560 | 129 | 0.96 |
| 24 | 60 | 4 | 134000 | 1996 | 2.28 | 125 | 66 | 127 | 0.95 |

*Polymerization conditions: partial pressure of ethylene = 4 kg/cm², initial hydrogen injection = 500 ml, polymerization time = 30 minutes, [Al]/[M]Ratio = 1/1000

As shown in the results of Table 2, when normal hexane was used, high activity, molecular weight, molecular weight distribution, and viscosity were observed, compared to the use of isobutane. Overall, as the polymerization temperature was increased, the activity was increased, and as the hydrogen content was increased, the molecular weight, molecular weight distribution, and viscosity were reduced.

In conclusion, the results of Tables 1 and 2 showed that higher activity and a wider range of physical properties were observed in normal hexane than isobutane. However, there were no problems in the preparation of products having desired physical properties, even though isobutane showed lower activity than normal hexane. In addition, upon polymerization with normal hexane, the mixture of solvent and wax makes the separation process difficult, whereas isobutane is advantageous in the separation process because of its rapid evaporation, which is attributed to higher evaporation temperature of normal hexane than isobutane.

### 2. Examples Using Supported Catalyst

### Examples 25-30

Ethylene polymerization was performed in the same manner as in Example 1, except that the supported catalyst of Preparation Example 3 and isobutane as a solvent were used.

At this time, polymerization reaction was performed for 60 minutes under the conditions of polymerization temperature of 60-70° C., ethylene injection of 10 kg/cm², and hydrogen injection of 1000-2000 ml. After termination of the polymerization, the reactor temperature was reduced to room temperature, and the polymer was recovered in the same manner as in Example 1. Then, the polymer was dried in a vacuum oven at 50° C. for 6 hours or longer.

### Examples 31-36

Ethylene polymerization was performed in the same manner as in Example 1, except that the supported catalyst of Preparation Example 3 and normal hexane as a solvent were used.

At this time, polymerization reaction was performed for 60 minutes under the conditions of polymerization temperature of 60-70° C., ethylene injection of 10 kg/cm², and hydrogen injection of 1000-2000 ml. After termination of the polymerization, the reactor temperature was reduced to room temperature, and the polymer was recovered in the same manner as in Example 1. Then, the polymer was dried in a vacuum oven at 50° C. for 6 hours or longer.

TABLE 3
Polymerization results with supported catalyst of Preparation
Example 3

| | | H₂ | Activity | | | | | | |
|---|---|---|---|---|---|---|---|---|---|
| | | Injection | g-PE/ | | | | | Softening | |
| | Temperature | amounts | g- | Mw | | Tm | Viscosity | Point | Density |
| Example | ° C. | % | Cat · hr | g/mol | MWD | ° C. | mPa · s | ° C. | g/cc |
| | Solvent: iso-butane | | | | | | | | |
| 25 | 60 | 1000 | 43000 | 11200 | 5.78 | 127 | 11000 | 133 | 0.97 |
| 26 | 60 | 1500 | 38000 | 9800 | 4.56 | 126 | 9800 | 132 | 0.97 |
| 27 | 60 | 2000 | 32000 | 8900 | 4.32 | 125 | 9100 | 131 | 0.96 |
| 28 | 70 | 1000 | 35000 | 15000 | 6.57 | 128 | 13200 | 135 | 0.97 |
| 29 | 70 | 1500 | 34000 | 12000 | 5.68 | 125 | 11500 | 134 | 0.97 |
| 30 | 70 | 2000 | 31000 | 10500 | 5.21 | 123 | 10900 | 132 | 0.97 |
| | Solvent: n-hexane | | | | | | | | |
| 31 | 60 | 1000 | 52000 | 12000 | 3.45 | 126 | 10500 | 134 | 0.97 |
| 32 | 60 | 1500 | 46000 | 11500 | 3.33 | 125 | 9500 | 131 | 0.96 |
| 33 | 60 | 2000 | 33000 | 10300 | 3.21 | 124 | 9300 | 130 | 0.96 |
| 34 | 70 | 1000 | 48000 | 9800 | 3.26 | 125 | 11500 | 133 | 0.97 |
| 35 | 70 | 1500 | 45000 | 8800 | 3.68 | 124 | 9700 | 132 | 0.97 |
| 36 | 70 | 2000 | 42000 | 8200 | 2.89 | 124 | 8200 | 132 | 0.97 |

*Polymerization conditions: partial pressure of ethylene = 10 kg/cm², polymerization time = 60 minutes

As shown in Table 3, when the polymerization was performed using the supported catalyst, physical properties could be controlled by the polymerization temperature and hydrogen content. The use of supported catalyst showed higher molecular weight and molecular weight distribution than the use of unsupported catalyst system, but the polymers were produced in a spherical particle form.

### Examples 37-42

Ethylene polymerization was performed in the same manner as in Example 1, except that the supported catalyst of Preparation Example 4 and isobutane as a solvent were used.

At this time, polymerization reaction was performed for 60 minutes under the conditions of polymerization temperature of 60-70° C., ethylene injection of 10 kg/cm², and hydrogen injection of 1000-2000 ml. After termination of the polymerization, the reactor temperature was reduced to room temperature, and the polymer was recovered. Then, the polymer was dried in a vacuum oven at 50° C. for 6 hours or longer.

### Polymerization Examples 43-48

Ethylene polymerization was performed in the same manner as in Example 1, except that the supported catalyst of Preparation Example 4 and normal hexane as a solvent were used.

At this time, polymerization reaction was performed for 60 minutes under the conditions of polymerization temperature of 60-70° C., ethylene injection of 10 kg/cm², and hydrogen injection of 1000-2000 ml. After termination of the polymerization, the reactor temperature was reduced to room temperature, and the polymer was recovered. Then, the polymer was dried in a vacuum oven at 50° C. for 6 hours or longer.

TABLE 4
Polymerization results with supported catalyst of Preparation
Example 4

| | | H₂ | Activity | | | | | | |
|---|---|---|---|---|---|---|---|---|---|
| | | Injection | g-PE/ | | | | | Softening | |
| | Temperature | amounts | g- | Mw | | Tm | Viscosity | Point | Density |
| Example | ° C. | % | Cat · hr | g/mol | MWD | ° C. | mPa · s | ° C. | g/cc |
| | Solvent: iso-butane | | | | | | | | |
| 37 | 60 | 1000 | 42000 | 3894 | 4.62 | 131 | 20156 | 136 | 0.97 |
| 38 | 60 | 1500 | 36000 | 2362 | 3.78 | 129 | 13617 | 131 | 0.96 |
| 39 | 60 | 2000 | 35000 | 2247 | 2.78 | 126 | 12737 | 127 | 0.95 |
| 40 | 70 | 1000 | 39000 | 3165 | 4.93 | 129 | 9100 | 133 | 0.97 |
| 41 | 70 | 1500 | 34000 | 1898 | 2.53 | 126 | 8600 | 131 | 0.96 |
| 42 | 70 | 2000 | 32000 | 1465 | 2.52 | 125 | 7880 | 129 | 0.96 |
| | Solvent: n-hexane | | | | | | | | |
| 43 | 60 | 1000 | 31600 | 5616 | 3.81 | 125 | 12000 | 128 | 0.96 |
| 44 | 60 | 1500 | 29800 | 3479 | 2.66 | 124 | 9570 | 124 | 0.95 |
| 45 | 60 | 2000 | 27000 | 3451 | 2.28 | 123 | 13700 | 128 | 0.95 |
| 46 | 70 | 1000 | 28000 | 8283 | 4.32 | 123 | 13700 | 128 | 0.96 |
| 47 | 70 | 1500 | 21400 | 7561 | 3.32 | 120 | 8500 | 127 | 0.96 |
| 48 | 70 | 2000 | 15800 | 6513 | 3.24 | 118 | 4207 | 123 | 0.95 |

*Polymerization conditions: partial pressure of ethylene = 10 kg/cm², polymerization time = 60 minutes

As shown in Table 4, low activity was observed, but narrow molecular weight distribution was observed, compared to the use of the supported catalyst system of Table 3.

Taken together, when polymerization was performed using the autoclave reactor and the metallocene catalyst, the molecular weight, viscosity, and softening point could be effectively controlled by polymerization temperature and hydrogen injection. Therefore, the commercial scale of the double loop reactor illustrated in FIGS. 1 and 2 can be sufficiently predicted, and a polyethylene wax can be provided in a more efficient manner than the conventional methods.

1. A method for preparing a polyethylene wax, comprising the step of polymerizing ethylene monomers in the presence of a metallocene catalyst in a loop reactor.

2. The method according to claim 1, wherein the loop reactor is a double loop reactor composed of a first reactor and a second reactor connected to each other.

3. The method according to claim 1, wherein a solvent of isobutane, normal hexane, or a mixture thereof is further used.

4. The method according to claim 1, wherein the metallocene catalyst includes a metallocene catalyst represented by the following Chemical Formula 1. 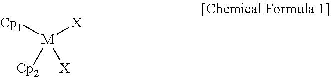
wherein M is a metal atom selected from titanium (Ti), zirconium (Zr), and hafnium (Hf), and Cp₁ and Cp₂ are each independently a cyclopentadienyl, indenyl or fluorenyl group; and X is a halogen atom, a C₁˜C₁₀ alkyl group, or a C₆˜C₂₀ aryl group.

5. The method according to claim 1, wherein the metallocene catalyst further includes an aluminium cocatalyst.

6. The method according to claim 5, wherein the metallocene catalyst has a molar ratio of aluminium of the aluminium cocatalyst to the metal of the Chemical Formula 1 of 1:500-1:2000.

7. The method according to claim 5, wherein the aluminium cocatalyst is alkyl aluminoxane where a C₁˜C₅ alkyl group is connected to aluminium.

8. The method according to claim 1, wherein the metallocene catalyst is an unsupported or supported catalyst.

9. The method according to claim 8, wherein the support used in the supported catalyst is selected from the group consisting of silica, alumina, magnesium chloride, zeolite, aluminium phosphate, and zirconia.

10. The method according to claim 8, wherein the support used in the supported catalyst is silica, and the silica is dehydrated silica having a specific surface area of 50 m²/g-500 m²/g, and a hydroxyl group of 0.5-3 number/cm².

11. The method according to claim 1, wherein the method is performed under the conditions of a polymerization temperature of 50-90° C., a hydrogen injection of 10% or less, a maximum reactor available pressure of 20-35 kg/cm², a maximum ethylene available pressure of 10 kg/cm², a polymerization time of 30˜180 minutes.

12. The method according to claim 1, wherein the method for preparing a polyethylene wax in a double loop reactor composed of a first reactor and a second reactor connected to each other includes the steps of:
polymerizing ethylene monomers and hydrogen in the presence of the metallocene catalyst and the solvent in the first reactor;
polymerizing a product produced by the above step and a solvent in a second reactor; and
separating a product of the second reactor in a separator.

13. The method according to claim 12, wherein the polymerization method further includes the step of reusing the solvent separated by the separator in the first and second reactors.

14. The method according to claim 12, wherein the polymerization method further includes the step of activating the catalyst by reaction of the metallocene catalyst and the cocatalyst, prior to the reaction step in the first reactor.
